SEQUENCE LISTING (8 sequences)

SEQ ID NO: 1 - protein, 332 aa, Rattus rattus
MATGGYRSSGSTTDFLEEWKAKREKMRAKQNPVGPGSSGGDPAAKSPAGPLAQTTAAGTSELNHGPAGAAAPAAPGPGALNCAHGSSALPRGAPGSRRPEDECPIAAGAAGAPASRGDEEEPDSAPEKGRSSGPSARKGKGQIEKRKLREKRRSTGVVNIPAAECLDEYEDDEAGQKERKREDAITQQNTIQNEAASLPDPGTSYLPQDPSRTVPGRYKSTISAPEEEILNRYPRTDRSGFSRHNRDTSAPANFASSSTLEKRIEDLEKEVLRERQENLRLTRLMQDKEEMIGKLKEEIDLLNRDLDDMEDENEQLKQENKTLLKVVGQLTR

SEQ ID NO: 2 - protein, 339 aa, Homo sapiens
ATGGYRTSSGLGGSTTDFLEEWKAKREKMRAKQNPPGPAPPGGGSSDAAGKPPAGALGTPAAAAANELNNNLPGGAPAAPAVPGPGGVNCAVGSAMLTRAAPGPRRSEDEPPAASASAAPPPQRDEEEPDGVPEKGKSSGPSARKGKGQIEKRKLREKRRSTGVVNIPAAECLDEYEDDEAGQKERKREDAITQQNTIQNEAVNLLDPGSSYLLQEPPRTVSGRYKSTTSVSEEDVSSRYSRTDRSGFPRYNRDANVSGTLVSSSTLEKKIEDLEKEVVRERQENLRLVRLMQDKEEMIGKLKEEIDLLNRDLDDIEDENEQLKQENKTLLKVVGQLTR

SEQ ID NO: 3 - protein, 234 aa, Homo sapiens
MQEPKCSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 234 aa, Artificial Sequence
  Modified Polypeptide Sequence, Synthetic
MQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 1701 bp, Artificial
  Modified Nucleotide Sequence, Synthetic
atgcaggagcctaagtcctccgacaaaactcacacatccccaccgtccccagcacctgaactcctggggggatcctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaatggcgaccggcggctatcggagcagcggcagcaccacggacttcctggaggagtggaaagcgaagcgcgagaagatgcgcgccaagcagaaccccgtgggcccgggttcgagcggcggggatccagccgccaagtcccctgcgggaccgctcgcccagactacggccgcggggacctcggaactcaaccacggccccgccggcgcggccgcacctgccgcccccgggccgggcgccctgaactgcgctcacggctcgtccgcgctgccccgcggggctcccggctcccggcggccggaggacgagtgtcctattgccgctggggccgcgggagcacccgcgtcccggggagacgaggaggagccggatagcgccccggagaagggccgcagctcggggcccagcgccaggaaaggcaaagggcagatcgagaagaggaagctgcgggagaagcgccgctccaccggcgtggtcaacatccccgcggcggagtgcttagatgagtacgaagatgacgaagcaggacagaaggaacggaagcgagaggatgctatcacacagcagaacaccatccagaatgaagctgcgagcctcccagatccaggaacctcctacctgccccaggacccgtcgagaacagtcccaggcagatacaaaagcacaatcagtgccccagaagaagaaatcttaaatagatatccccgaacagatagaagtggcttcagtagacacaacagagataccagtgcgcctgctaacttcgcttcaagtagcaccttggaaaagagaattgaagatcttgagaaggaagtcttgagagaaaggcaagaaaaccttcgacttacgaggctgatgcaagataaagaagaaatgattggaaaactcaaggaagagattgatttgttaaatagagacctcgatgacatggaagacgaaaacgagcaactaaagcaggaaaataaaactcttttgaaagttgttgggcagctgacaaggtag

SEQ ID NO: 6 - protein, 566 aa, Artificial
  Modified Polypeptide Sequence, Synthetic
MQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMATGGYRSSGSTTDFLEEWKAKREKMRAKQNPVGPGSSGGDPAAKSPAGPLAQTTAAGTSELNHGPAGAAAPAAPGPGALNCAHGSSALPRGAPGSRRPEDECPIAAGAAGAPASRGDEEEPDSAPEKGRSSGPSARKGKGQIEKRKLREKRRSTGVVNIPAAECLDEYEDDEAGQKERKREDAITQQNTIQNEAASLPDPGTSYLPQDPSRTVPGRYKSTISAPEEEILNRYPRTDRSGFSRHNRDTSAPANFASSSTLEKRIEDLEKEVLRERQENLRLTRLMQDKEEMIGKLKEEIDLLNRDLDDMEDENEQLKQENKTLLKVVGQLTR

SEQ ID NO: 7 - DNA, 1725 bp, Artificial
  Modified nucleic acid sequence, Synthetic
atgcaggagcctaagtcctccgacaaaactcacacatccccaccgtccccagcacctgaactcctggggggatcctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaatggcgaccggtggctaccggaccagcagcggcctcggcggcagcaccacagacttcctggaggagtggaaggcgaaacgcgagaagatgcgcgccaagcagaaccccccgggcccggcccccccgggagggggcagcagcgacgccgctgggaagccccccgcgggggctctgggcaccccggcggccgccgctgccaacgagctcaacaacaacctcccgggcggcgcgccggccgcacctgccgtccccggtcccgggggcgtgaactgcgcggtcggctccgccatgctgacgcgggcggcccccggcccgcggcggtcggaggacgagcccccagccgcctctgcctcggctgcaccgccgccccagcgtgacgaggaggagccggacggcgtcccagagaagggcaagagctcgggccccagtgccaggaaaggcaaggggcagatcgagaagaggaagctgcgggagaagcggcgctccaccggcgtggtcaacatccctgccgcagagtgcttagatgagtacgaagatgatgaagcagggcagaaagagcggaaacgagaagatgcaattacacaacagaacactattcagaatgaagctgtaaacttactagatccaggcagttcctatctgctacaggagccacctagaacagtttcaggcagatataaaagcacaaccagtgtctctgaagaagatgtctcaagtagatattctcgaacagatagaagtgggttccctagatataacagggatgcaaatgtttcaggtactctggtttcaagtagcacactggaaaagaaaattgaagatcttgaaaaggaagtagtaagagaaagacaagaaaacctaagacttgtgagactgatgcaagataaagaggaaatgattggaaaactcaaagaagaaattgatttattaaatagagacctagatgacatagaagatgaaaatgaacagctaaagcaggaaaataaaactcttttgaaagttgtgggtcagctgaccaggtag

SEQ ID NO: 8 - protein, 574 aa, Artificial
  Modified polypeptide sequence, Synthetic
MQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMATGGYRTSSGLGGSTTDFLEEWKAKREKMRAKQNPPGPAPPGGGSSDAAGKPPAGALGTPAAAAANELNNNLPGGAPAAPAVPGPGGVNCAVGSAMLTRAAPGPRRSEDEPPAASASAAPPPQRDEEEPDGVPEKGKSSGPSARKGKGQIEKRKLREKRRSTGVVNIPAAECLDEYEDDEAGQKERKREDAITQQNTIQNEAVNLLDPGSSYLLQEPPRTVSGRYKSTTSVSEEDVSSRYSRTDRSGFPRYNRDANVSGTLVSSSTLEKKIEDLEKEVVRERQENLRLVRLMQDKEEMIGKLKEEIDLLNRDLDDIEDENEQLKQENKTLLKVVGQLTR

(19) United States
(12) Patent Application Publication
(10) Pub. No.: US 2022/0153805 A1
(43) Pub. Date: May 19, 2022

(54) A Novel Prostate Apoptosis Response-4 (Par-4) Protein Entity with an Extended Duration of Action for Therapeutic Treatment of Cancer

(71) Applicant: University of Kentucky Research Foundation, Lexington, KY (US)

(72) Inventors: Chang-Guo Zhan, Lexington, KY (US); Fang Zheng, Lexington, KY (US); Vivek M. Rangnekar, Lexington, KY (US); Kyungbo Kim, Lexington, KY (US)

(21) Appl. No.: 17/440,544

(22) PCT Filed: Apr. 10, 2020
(86) PCT No.: PCT/US2020/027623

§ 371 Date: Sep. 17, 2021

Related U.S. Application Data
(60) Provisional application No. 62/832,155, filed on Apr. 10, 2019.

(51) Int. Cl. C07K 14/705; A61P 35/04

(52) U.S. Cl. CPC C07K 14/705; A61K 38/00; C07K 2319/30; A61P 35/04

(57) ABSTRACT

Disclosed herein are polypeptide molecules having Prostate apoptosis response-4 (Par-4) pro-apoptotic activity in cancer cells and enhanced biological half-life, and related methods. Specifically, the disclosure provides a composition comprising Par-4 polypeptide fusion protein, and a method of inducing apoptosis in a cancer cell using the composition, wherein the cancer cell is metastatic.

### RELATED APPLICATIONS

This application claims priority from U.S. Provisional Application Ser. No. 62/832,155, filed Apr. 10, 2019, the entire disclosure of which is incorporated herein by this reference.

### TECHNICAL FIELD

The presently-disclosed subject matter generally relates to polypeptide molecules having Prostate apoptosis response-4 (Par-4) pro-apoptotic activity in cancer cells and enhanced biological half-life. The presently-disclosed subject matter also relates to nucleic acid molecules encoding such polypeptide molecules, methods of making recombinant polypeptide molecules, compositions including such polypeptide molecules, methods of inducing apoptosis in a cancer cell, and methods of treating cancer.

### INTRODUCTION

Prostate apoptosis response-4 (Par-4) is a tumor suppressor protein expressed ubiquitously in a number of tissues. In 1994, the Par-4 gene was first discovered as an early apoptotic gene in a rat prostate cancer cell line incubated with ionomycin for apoptotic cell death. (1,2) It was demonstrated that overexpression of Par-4 is sufficient to elicit apoptotic cell death in most cancer cells. (3) In line with this observation, the Par-4 gene has been reported to be mutated in endometrial cancer, (4) and significantly down-regulated in many different types of cancer including renal cell carcinoma, (5) breast cancer, (6,7) gastric and pancreatic cancer, (8) glioblastoma, (9) and neuroblastoma. (10) Par-4 downregulation is associated with tumor recurrence and diminished patient survival. (7)

The core domain of Par-4 (amino-acid residues 145-204 in human Par-4; and 137-195 in rat Par-4), known as Selective for Apoptosis in Cancer cells (SAC), serves as the effector domain responsible for its pro-apoptotic activity. (11) Notably, this domain is 100% conserved in mouse, rat, and human homologs, which implies that Par-4 plays a critical role in the surveillance against tumors. (11) Indeed, mature Par-4 protein and its SAC domain both are capable of inducing apoptotic cell death through both an intrinsic pathway (activated by intrinsic stimuli such as biochemical stress or DNA damage, and mainly modulated by Bcl-2 and Bax)(12) and extrinsic pathway (activated in response to external stimuli such as Fas ligand). (13)

At first, it was believed that Par-4 protein localizes and acts only in the cytoplasm and the nucleus for apoptosis induction, (1,14) but subsequent studies revealed that Par-4 protein can be secreted to the extracellular space for action. (2) Extracellular Par-4 protein can induce apoptosis via FADD, caspase-8 and -3 activation following binding to the stress response protein, i.e. glucose regulated protein 78 (GRP78), expressed on the surface of cancer cells. (2)

It has also been demonstrated that exposure to purified recombinant Par-4 protein not only induces apoptosis in multiple types of cancer cells, but also inhibits tumor growth in vivo. (3,15) Therefore, the previous Par-4 related drug discovery efforts have focused on development of small-molecule drugs that can facilitate Par-4 secretion from normal cells for Par-4-dependent inhibition of tumor growth. Arylquin 1(16) and chloroquine (CQ), an anti-malarial drug, (17) have been discovered as a strong inducer of Par-4 secretion from normal or cancer cells.

Recombinant wild-type Par-4 stays in the circulatory system of mice only for a very short period of time. Thus, the tumor-suppressing activity of secreted Par-4 may be diminished due to its limited serum persistence in vivo. It is well recognized that the practical therapeutic efficacy of a protein drug can be greatly increased by improving its pharmacokinetic (PK) profile. (18-24)

Accordingly, there remains a need in the art for a composition having improved biological half-life, in vivo, while maintaining the beneficial biological activity of Par-4.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes polypeptide molecules having Prostate apoptosis response-4 (Par-4) activity and enhanced biological half-life. The presently-disclosed subject matter also includes nucleic acid molecules encoding such polypeptide molecules, methods of making recombinant polypeptide molecules, compositions including such polypeptide molecules, methods of inducing apoptosis in a cancer cell, and methods of treating cancer.

Polypeptide molecules disclosed herein have Par-4 activity and enhanced biological half-life, relative to wild-type Par-4. In some embodiments, the polypeptide molecule includes a Par-4 polypeptide portion and a second polypeptide molecule, provided as a fusion protein.

In some embodiments, the Par-4 polypeptide portion is a polypeptide comprising the sequence of SEQ ID NO: 1 or SEQ ID NO: 2. In some embodiments, the Par-4 polypeptide portion is a polypeptide comprising a modification relative to the sequence of SEQ ID NO: 1 or SEQ ID NO: 2. In some embodiments, the Par-4 polypeptide portion is a polypeptide comprising a deletion relative to the sequence of SEQ ID NO: 1 or SEQ ID NO: 2. In some embodiments, the Par-4 polypeptide portion is a polypeptide comprising an insertion relative to the sequence of SEQ ID NO: 1 or SEQ ID NO: 2. In some embodiments, the Par-4 polypeptide portion is a polypeptide comprising a substitution relative to the sequence of SEQ ID NO: 1 or SEQ ID NO: 2.

In some embodiments, the Par-4 polypeptide component is a polypeptide comprising a fragment of the sequence of SEQ ID NO: 1 or SEQ ID NO: 2. In some embodiments, the Par-4 polypeptide component is a polypeptide comprising a variant of the sequence of SEQ ID NO: 1 or SEQ ID NO: 2.

In some embodiments, the second polypeptide molecule comprises a fragment crystallizable of immunoglobulin G1(Fc) polypeptide comprising the sequence of SEQ ID NO: 3 or SEQ ID NO: 4. In some embodiments, the second polypeptide molecule is a polypeptide comprising a modification relative to the sequence of SEQ ID NO: 3 or SEQ ID NO: 4. In some embodiments, the second polypeptide molecule is a polypeptide comprising a deletion relative to the sequence of SEQ ID NO: 3 or SEQ ID NO: 4. In some embodiments, the second polypeptide molecule is a polypeptide comprising a insertion relative to the sequence of SEQ ID NO: 3 or SEQ ID NO: 4. In some embodiments, the second polypeptide molecule is a polypeptide comprising a substitution relative to the sequence of SEQ ID NO: 3 or SEQ ID NO: 4.

In some embodiments, the second polypeptide molecule is a fragment the sequence of SEQ ID NO: 3 or SEQ ID NO: 4. In some embodiments, the second polypeptide molecule is a variant the sequence of SEQ ID NO: 3 or SEQ ID NO: 4.

The presently-disclosed subject matter also includes a method of inducing apoptosis in a cancer cell, which involves contacting the cancer cell with a polypeptide molecule as disclosed herein. In some embodiments, the cancer cell is metastatic.

The presently-disclosed subject matter also includes a method of treating cancer in a subject, which involves administering a polypeptide molecule as disclosed herein to a subject in need thereof.

The presently-disclosed subject matter further includes nucleic acid molecules. Nucleic acid molecules disclosed herein include a nucleotide sequence, or complement thereof, encoding a polypeptide molecule as disclosed herein above. In this regard, the nucleic acid molecules encode a polypeptide molecule having Par-4 activity and enhanced biological half-life, relative to wild-type Par-4. The nucleic acid molecules include a sequence encoding a Par-4 polypeptide as disclosed herein, operably linked with a sequence encoding a second polypeptide molecule as disclosed herein. In some embodiments, the nucleic acid molecule encodes a polypeptide molecule includes a Par-4 polypeptide portion and a second polypeptide molecule, provided as a fusion protein.

The presently disclosed subject matter further includes vectors comprising a nucleic acid molecule as disclosed herein. The presently-disclosed subject matter further includes a cell comprising a vector comprising a nucleic acid molecule as disclosed herein. The presently-disclosed subject matter further includes a method of making a polypeptide molecule as disclosed herein using a nucleic acid molecule as disclosed herein.

The presently-disclosed subject matter further includes compositions, such as pharmaceutical compositions, comprising a polypeptide molecule as disclosed herein, having Par-4 activity and enhanced biological half-life, relative to wild-type Par-4. The composition can include a pharmaceutically-acceptable carrier and provide for the stability of the polypeptide molecule, as will be understood by those of ordinary skill in the art.

The presently-disclosed subject matter also includes a method of inducing apoptosis in a cancer cell, which involves contacting the cancer cell with a composition as disclosed herein. In some embodiments, the cancer cell is metastatic.

The presently-disclosed subject matter also includes a method of treating cancer in a subject, which involves administering a composition as disclosed herein to a subject in need thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 shows the limited serum persistence of recombinant Par-4 proteins in mice. (A) SDS-PAGE of the purified TRX-fused or hexa-histidine-tagged Par-4 (TRX-Par-4 and 6×His-Par-4, respectively). Mice were injected intravenously with 5 mg/kg TRX-Par-4 or 6×His-Par-4. Relative serum concentrations of TRX-Par-4 (B) and 6×His-Par-4 (C) were evaluated by western blotting. The recombinant Par-4 proteins were detected with antibody against Par-4 and visualized by chemiluminescence. The light chain of mouse IgG1 was used as an internal loading control. The two panels are representative blots.

FIG. 2 shows the preparation of Par-4Ex. (A) The schematic presentation of Par-4Ex or Fc(M1)-Par-4. (B) Western blot analysis of Par-4 protein in bacterial extract transformed with pET-22b(+)/6×his-Par-4 and induced with IPTG. The soluble fraction (S) of the bacterial extract was separated from the insoluble fraction (I) by centrifugation before immunoblotting. (C) SDS-PAGE of the purified Par-4Ex protein. Soluble Par-4Ex protein was isolated by protein A chromatography (Protein A), followed by an additional ion-exchange chromatographic step (Ion exchanger) to achieve the purity required.

FIG. 3 shows Recombinant Par-4 and Par-4Ex proteins elicit apoptosis in E0771 (murine breast cancer cell line) cells. (A) SDS-PAGE of the purified Par-4Ex. (B) The cells were treated with vehicle, or purified Par-4 (6×His-Par-4) or Par-4Ex (100 nM each). 24 h after treatment, the cells were scored for apoptosis by immunocytochemistry (ICC) for caspase 3 activity. Results represent the average of triplicates and the values are expressed as mean±S.D. Asterisk (*) indicates the difference is statistically significant (p<0.05) by Student's t-test

FIG. 4 shows serum concentration (%) versus time profiles of recombinant Par-4 and Par-4Ex proteins in mice. E. coli-derived Par-4Ex (▪) or 6×His-Par-4 (□) was administered via i.v. infusion at 5 mg/kg and the serum protein concentrations were determined by ELISA. Results represent the average of triplicates per group and shown as mean±standard error.

FIG. 5 shows recombinant Par-4 protein suppresses the metastatic growth of tumor (E0771). The cells (1.5×10⁵ cells) were administered into tail vein in B6C3H mice (n=5). (A) 5 h after administration, vehicle, or 250 μg purified Par-4 (6×His-Par-4) or Par-4Ex was injected through tail vein every alternate day for 12 days (for a total of 1500 μg of protein/mouse). (B) 5 h after administration, vehicle, or 150 or 75 μg purified Par-4Ex was injected through tail vein every alternate day for 12 days (for a total of 900 and 450 μg of protein/mouse). Four weeks later, the mice were euthanized, and the number of the lung nodules were then counted. The data are expressed as the mean±SEM. The single and double asterisks indicate p<0.05 and p<0.01, respectively.

### BRIEF DESCRIPTION OF THE SEQUENCE LISTING

SEQ ID NO: 1 includes an amino acid sequence for a Rat Par-4.

SEQ ID NO: 2 includes an amino acid sequence for a Human Par-4.

SEQ ID NO: 3 includes an amino acid sequence for Fc Polypeptide.

SEQ ID NO: 4 includes an amino acid sequence for a modified Fc Polypeptide.

SEQ ID NO: 5 includes a nucleic acid sequence encoding a modified Rat Par-4 (Par-4Ex) polypeptide;

SEQ ID NO: 6 includes an amino acid sequence for a modified Rat Par-4 (Par-4Ex) polypeptide;

SEQ ID NO: 7 includes a nucleic acid sequence encoding a modified Human Par-4 (Par-4Ex) polypeptide; and

SEQ ID NO: 8 includes an amino acid sequence for a modified Human Par-4 (Par-4Ex) polypeptide.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes polypeptide molecules having Prostate apoptosis response-4 (Par-4) activity and enhanced biological half-life. The presently-disclosed subject matter also includes nucleic acid molecules encoding such polypeptide molecules, methods of making recombinant polypeptide molecules, compositions including such polypeptide molecules, methods of inducing apoptosis in a cancer cell, and methods of treating cancer.

Polypeptide Molecules

Polypeptide molecules disclosed herein have Par-4 activity and enhanced biological half-life, relative to wild-type Par-4. In some embodiments, the polypeptide molecule includes a Par-4 polypeptide portion and a second polypeptide molecule, provided as a fusion protein.

In some embodiments, the Par-4 polypeptide portion is a polypeptide comprising the sequence of SEQ ID NO: 1 or SEQ ID NO: 2. In some embodiments, the Par-4 polypeptide portion is a polypeptide comprising a modification relative to the sequence of SEQ ID NO: 1 or SEQ ID NO: 2. In some embodiments, the Par-4 polypeptide portion is a polypeptide comprising a deletion relative to the sequence of SEQ ID NO: 1 or SEQ ID NO: 2. In some embodiments, the Par-4 polypeptide portion is a polypeptide comprising an insertion relative to the sequence of SEQ ID NO: 1 or SEQ ID NO: 2. In some embodiments, the Par-4 polypeptide portion is a polypeptide comprising a substitution relative to the sequence of SEQ ID NO: 1 or SEQ ID NO: 2.

In some embodiments, the Par-4 polypeptide component is a polypeptide comprising a fragment of the sequence of SEQ ID NO: 1 or SEQ ID NO: 2. In some embodiments, the Par-4 polypeptide component is a polypeptide comprising a variant of the sequence of SEQ ID NO: 1 or SEQ ID NO: 2.

As used herein, a “modification” is in reference to modification of a sequence of amino acids of a polypeptide or a sequence of nucleotides in a nucleic acid molecule and includes deletions, insertions, and substitutions of amino acids and nucleotides, respectively. Methods of modifying a polypeptide are routine to those of skill in the art, such as by using recombinant DNA methodologies or direct synthesis.

As used herein, “deletion,” when referring to a nucleic acid molecule or polypeptide, refers to the deletion of one or more nucleotides from either termini of the nucleic acid molecule or deletion of one or more amino acids from either termini of the polypeptide compared to a reference sequence, such as a wild-type sequence.

As used herein, “insertion” when referring to a nucleic acid molecule or polypeptide, describes the inclusion of one or more additional nucleotides in the nucleic acid molecule or one or more amino acids in the polypeptide, within a reference sequence, such as a wild-type sequence. Thus, a molecule that contains one or more insertions compared to a wild-type sequence, contains one or more additional residues within the linear length of the sequence.

As used herein, “additions,” to nucleic acid molecules and polypeptides describe addition of nucleotides or amino acids onto either termini compared to a reference nucleic acid molecule or polypeptide.

As used herein, “substitution” refers to the replacing of one or more nucleotides or amino acids in a reference sequence with an alternative nucleotide or amino acid, without changing the length (as described in numbers of residues) of the molecule. Thus, one or more substitutions in a molecule does not change the number of amino acid residues or nucleotides of the molecule.

The terms “polypeptide fragment” or “fragment”, when used in reference to a reference polypeptide, refers to a polypeptide in which amino acid residues are deleted as compared to the reference polypeptide itself. A fragment can also be a “functional fragment,” in which case the fragment retains some or all of the activity of the reference polypeptide as described herein.

The terms “polypeptide variant” refer to an amino acid sequence that is different from the reference polypeptide by one or more amino acid substitutions, e.g., one or more amino acid substitutions. A variant of a reference polypeptide also refers to a variant of a fragment of the reference polypeptide, for example, a fragment wherein one or more amino acid substitutions have been made relative to the reference polypeptide. A variant can also be a “functional variant,” in which the variant retains some or all of the activity of the reference protein as described herein.

As used herein, an “activity” of a polypeptide refers to any activity exhibited by wild-type Par-4, for example, binding to the stress response protein glucose regulated protein 78 (GRP78), apoptotic activity in a cancer cell, and suppressing tumor activity. Activity of a modified polypeptide can be any level of percentage of activity of the unmodified polypeptide, including but not limited to, 50% of the activity, 60%, 70%, 80%, 90%, 91%, 92%, 93%, 94%, 95%, 96%, 97%, 98%, 99%, 100%, 200%, 300%, 400%, 500%, or more of activity compared to the unmodified polypeptide.

In some embodiments, the Par-4 polypeptide portion includes a sequence having at least 85%, 86%, 87%, 88%, 89%, 90%, 91%, 92%, 93%, 94%, 95%, 96%, 97%, 98%, or 99% homology to the sequence of SEQ ID NO: 1 or SEQ ID NO: 2. In some embodiments, the polypeptide molecule includes a Par-4 polypeptide portion including a fragment of the sequence of SEQ ID NO: 1 or SEQ ID NO: 2 having 100% homology to the sequence of SEQ ID NO: 1 or SEQ ID NO: 2 in a region including a selective for apoptosis in cancer cells (SAC) core domain. In some embodiments, the polypeptide molecule includes a Par-4 polypeptide portion including a fragment of the sequence of SEQ ID NO: 1 or SEQ ID NO: 2 wherein 1, 2, 3, 4, 5, 6, 7, 8, 9, or 10 residues have been deleted relative to the sequence of SEQ ID NO: 1 or SEQ ID NO: 2.

Sequence “identity” or “homology” has an art-recognized meaning and the percentage of sequence identity between two nucleic acid molecules or polypeptide molecules, or regions thereof, can be calculated using published techniques. Sequence identity can be measured along the full length of a polynucleotide or polypeptide, or along a region of the molecule. While there exist a number of methods to measure identity between two polynucleotide or polypeptides, the term “identity” is well known to skilled artisans (Carrillo, H. & Lipman, D., SIAM J Applied Math 48:1073 (1988)).

Sequence identity compared along the full length of two polynucleotides or polypeptides refers to the percentage of identical nucleotide or amino acid residues along the full-length of the molecule. For example, if a polypeptide A has 100 amino acids and polypeptide B has 95 amino acids, which are identical to amino acids 1-95 of polypeptide A, then polypeptide B has 95% identity when sequence identity is compared along the full length of a polypeptide A compared to full length of polypeptide B. Alternatively, sequence identity between polypeptide A and polypeptide B can be compared along a region, such as a 20 amino acid analogous region, of each polypeptide. In this case, if polypeptide A and B have 20 identical amino acids along that region, the sequence identity for the regions is 100%. For example, in some embodiments, the Par-4 polypeptide portion of the polypeptide molecule described herein can have 100% identity to wild type Par-4 in the core domain of Par-4 (amino-acid residues 145-204 in human Par-4; and 137-195 in rat Par-4), known as Selective for Apoptosis in Cancer cells (SAC).

As noted herein above, in addition to the Par-4 polypeptide portion, the polypeptide molecule includes a second polypeptide molecule, provided with the Par-4 polypeptide portion a as a fusion protein.

In some embodiments, the second polypeptide molecule comprises a fragment crystallizable of immunoglobulin G1(Fc) polypeptide comprising the sequence of SEQ ID NO: 3 or SEQ ID NO: 4. In some embodiments, the second polypeptide molecule is a polypeptide comprising a modification relative to the sequence of SEQ ID NO: 3 or SEQ ID NO: 4. In some embodiments, the second polypeptide molecule is a polypeptide comprising a deletion relative to the sequence of SEQ ID NO: 3 or SEQ ID NO: 4. In some embodiments, the second polypeptide molecule is a polypeptide comprising a insertion relative to the sequence of SEQ ID NO: 3 or SEQ ID NO: 4. In some embodiments, the second polypeptide molecule is a polypeptide comprising a substitution relative to the sequence of SEQ ID NO: 3 or SEQ ID NO: 4.

In some embodiments, the second polypeptide molecule is a fragment the sequence of SEQ ID NO: 3 or SEQ ID NO: 4. In some embodiments, the second polypeptide molecule is a variant the sequence of SEQ ID NO: 3 or SEQ ID NO: 4.

In some embodiments, the second polypeptide molecule including a sequence having at least 85%, 86%, 87%, 88%, 89%, 90%, 91%, 92%, 93%, 94%, 95%, 96%, 97%, 98%, or 99% homology to the sequence of SEQ ID NO: 3 or SEQ ID NO: 4. In some embodiments, the second polypeptide molecule includes a fragment of the sequence of SEQ ID NO: 3 or SEQ ID NO: 4, wherein 1, 2, 3, 4, 5, 6, 7, 8, 9, or 10 residues have been deleted relative to the sequence of SEQ ID NO: 3 or SEQ ID NO: 4. In some embodiments, the second polypeptide molecule includes a variant of SEQ ID NO: 3 or SEQ ID NO: 4, wherein cysteine residues of the polypeptide have been mutated relative to the sequence of SEQ ID NO: 3 or SEQ ID NO: 4. In some embodiments, the second polypeptide molecule includes a variant of a fragment of SEQ ID NO: 3 or SEQ ID NO: 4, wherein cysteine residues of the polypeptide have been mutated relative to the sequence of SEQ ID NO: 3 or SEQ ID NO: 4, and wherein 1, 2, 3, 4, 5, 6, 7, 8, 9, or 10 residues have been deleted relative to the sequence of SEQ ID NO: 3 or SEQ ID NO: 4. In some embodiments, the second polypeptide molecule includes a variant of SEQ ID NO: 3 or SEQ ID NO: 4 comprising the substitutions C6S, C12S, and C15S. In some embodiments, the second polypeptide molecule includes a variant of a fragment SEQ ID NO: 3 or SEQ ID NO: 4 comprising the substitutions C6S, C12S, and C15S, and wherein 1, 2, 3, 4, 5, 6, 7, 8, 9, or 10 residues have been deleted relative to the sequence of SEQ ID NO: 3 or SEQ ID NO: 4.

In some embodiments, the polypeptide molecule includes an embodiment of the second polypeptide molecule as disclosed herein positioned proximal to the N-terminus of an embodiment of the Par-4 polypeptide portion as disclosed herein.

The terms “amino-terminal,” or “N-terminal,” and “carboxyl-terminal,” or “C-terminal,” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

In some embodiments, the polypeptide molecule as disclosed herein comprises the sequence of SEQ ID NO: 6. In some embodiments, the polypeptide molecule as disclosed herein comprises the sequence of SEQ ID NO: 8.

The presently-disclosed subject matter also includes a method of inducing apoptosis in a cancer cell, which involves contacting the cancer cell with a polypeptide molecule as disclosed herein. In some embodiments, the cancer cell is metastatic.

The presently-disclosed subject matter also includes a method of treating cancer in a subject, which involves administering a polypeptide molecule as disclosed herein to a subject in need thereof.

As used herein, the terms “treatment” or “treating” relate to any treatment of a cancer including but not limited to prophylactic treatment to reduce severity of the cancer, as well as therapeutic treatment. In this regard, in is understood that treatment can involve curing the cancer, or substantially curing the cancer, or ameliorating at least one symptom of the cancer, or reducing the severity of the cancer.

As used herein, the term “subject” refers to a target of administration. The subject of the herein disclosed methods can be a vertebrate, such as a mammal. The subject of the herein disclosed methods can be a human or non-human. Thus, veterinary therapeutic uses are provided in accordance with the presently disclosed subject matter.

Nucleic Acid Molecules

The presently-disclosed subject matter further includes nucleic acid molecules. Nucleic acid molecules disclosed herein include a nucleotide sequence, or complement thereof, encoding a polypeptide molecule as disclosed herein above. In this regard, the nucleic acid molecules encode a polypeptide molecule having Par-4 activity and enhanced biological half-life, relative to wild-type Par-4. The nucleic acid molecules include a sequence encoding a Par-4 polypeptide as disclosed herein, operably linked with a sequence encoding a second polypeptide molecule as disclosed herein. In some embodiments, the nucleic acid molecule encodes a polypeptide molecule includes a Par-4 polypeptide portion and a second polypeptide molecule, provided as a fusion protein.

The term “complementary” refers to two nucleotide sequences (a sequence and complement thereof) that comprise antiparallel nucleotide sequences capable of pairing with one another upon formation of hydrogen bonds between the complementary base residues in the antiparallel nucleotide sequences. As is known in the art, the nucleic acid sequences of two complementary strands are the reverse complement of each other when each is viewed in the 5′ to 3′ direction.

As used herein, “operably linked” with reference to nucleic acid molecule sequences, means that the nucleic acid molecule sequences are functionally related to each other. For example, a first nucleic acid molecule encoding a first polypeptide can be operably linked to a second nucleic acid molecule encoding a second polypeptide, whereby the nucleic acid molecules can be transcribed and translated to express a functional fusion protein.

In some embodiments, the nucleic acid molecule includes a sequence encoding a polypeptide comprising the sequence of SEQ ID NO: 1 or SEQ ID NO: 2. In some embodiments, the nucleic acid molecule includes a sequence encoding a polypeptide comprising a modification relative to the sequence of SEQ ID NO: 1 or SEQ ID NO: 2. In some embodiments, the nucleic acid molecule includes a sequence encoding a polypeptide comprising a deletion relative to the sequence of SEQ ID NO: 1 or SEQ ID NO: 2. In some embodiments, the nucleic acid molecule includes a sequence encoding a polypeptide comprising an insertion relative to the sequence of SEQ ID NO: 1 or SEQ ID NO: 2. In some embodiments, the nucleic acid molecule includes a sequence encoding a polypeptide comprising a substitution relative to the sequence of SEQ ID NO: 1 or SEQ ID NO: 2.

In some embodiments, the nucleic acid molecule includes a sequence encoding a polypeptide comprising a fragment of the sequence of SEQ ID NO: 1 or SEQ ID NO: 2. In some embodiments, the nucleic acid molecule includes a sequence encoding a polypeptide comprising a variant of the sequence of SEQ ID NO: 1 or SEQ ID NO: 2.

In some embodiments, the nucleic acid molecule includes a sequence encoding a second polypeptide molecule comprises a fragment crystallizable of immunoglobulin G1(Fc) polypeptide comprising the sequence of SEQ ID NO: 3 or SEQ ID NO: 4. In some embodiments, the nucleic acid molecule includes a sequence encoding a polypeptide comprising a modification relative to the sequence of SEQ ID NO: 3 or SEQ ID NO: 4. In some embodiments, the nucleic acid molecule includes a sequence encoding a polypeptide comprising a deletion relative to the sequence of SEQ ID NO: 3 or SEQ ID NO: 4. In some embodiments, the nucleic acid molecule includes a sequence encoding a polypeptide comprising a insertion relative to the sequence of SEQ ID NO: 3 or SEQ ID NO: 4. In some embodiments, the nucleic acid molecule includes a sequence encoding a polypeptide comprising a substitution relative to the sequence of SEQ ID NO: 3 or SEQ ID NO: 4.

In some embodiments, the nucleic acid molecule includes a sequence encoding a fragment the sequence of SEQ ID NO: 3 or SEQ ID NO: 4. In some embodiments, the nucleic acid molecule includes a sequence encoding a variant the sequence of SEQ ID NO: 3 or SEQ ID NO: 4.

In some embodiments, the nucleic acid molecule includes a sequence encoding a sequence having at least 85%, 86%, 87%, 88%, 89%, 90%, 91%, 92%, 93%, 94%, 95%, 96%, 97%, 98%, or 99% homology to the sequence of SEQ ID NO: 3 or SEQ ID NO: 4. In some embodiments, the nucleic acid molecule includes a sequence encoding a fragment of the sequence of SEQ ID NO: 3 or SEQ ID NO: 4, wherein 1, 2, 3, 4, 5, 6, 7, 8, 9, or 10 residues have been deleted relative to the sequence of SEQ ID NO: 3 or SEQ ID NO: 4. In some embodiments, the nucleic acid molecule includes a sequence encoding a variant of SEQ ID NO: 3 or SEQ ID NO: 4, wherein cysteine residues of the polypeptide have been mutated relative to the sequence of SEQ ID NO: 3 or SEQ ID NO: 4. In some embodiments, the nucleic acid molecule includes a sequence encoding a variant of a fragment of SEQ ID NO: 3 or SEQ ID NO: 4, wherein cysteine residues of the polypeptide have been mutated relative to the sequence of SEQ ID NO: 3 or SEQ ID NO: 4, and wherein 1, 2, 3, 4, 5, 6, 7, 8, 9, or 10 residues have been deleted relative to the sequence of SEQ ID NO: 3 or SEQ ID NO: 4. In some embodiments, the nucleic acid molecule includes a sequence encoding a variant of SEQ ID NO: 3 or SEQ ID NO: 4 comprising the substitutions C6S, C12S, and C15S. In some embodiments, the nucleic acid molecule includes a sequence encoding a variant of a fragment SEQ ID NO: 3 or SEQ ID NO: 4 comprising the substitutions C6S, C12S, and C15S, and wherein 1, 2, 3, 4, 5, 6, 7, 8, 9, or 10 residues have been deleted relative to the sequence of SEQ ID NO: 3 or SEQ ID NO: 4.

In some embodiments, the nucleic acid molecule includes a sequence encoding an embodiment of the second polypeptide molecule as disclosed herein positioned proximal to the N-terminus of a sequence encoding an embodiment of the Par-4 polypeptide portion as disclosed herein.

In some embodiments, the nucleic acid molecule as disclosed herein comprises the sequence of SEQ ID NO: 5, or a complement thereof. In some embodiments, the nucleic acid molecule as disclosed herein comprises the sequence of SEQ ID NO: 7, or a complement thereof.

The presently disclosed subject matter further includes vectors comprising a nucleic acid molecule as disclosed herein. The presently-disclosed subject matter further includes a cell comprising a vector comprising a nucleic acid molecule as disclosed herein. The presently-disclosed subject matter further includes a method of making a polypeptide molecule as disclosed herein using a nucleic acid molecule as disclosed herein.

As used herein, a “vector” is a replicable nucleic acid molecule from which one or more heterologous proteins can be expressed when the vector is transformed into an appropriate host cell. A “host cell” is a cell that is used in to receive, maintain, reproduce, and amplify a vector. A host cell also can be used to express the polypeptide encoded by the vector.

Compositions

The presently-disclosed subject matter further includes compositions, such as pharmaceutical compositions, comprising a polypeptide molecule as disclosed herein, having Par-4 activity and enhanced biological half-life, relative to wild-type Par-4. The composition can include a pharmaceutically-acceptable carrier and provide for the stability of the polypeptide molecule, as will be understood by those of ordinary skill in the art.

The compositions comprise a polypeptide molecule including a Par-4 polypeptide portion and a second polypeptide molecule, provided as a fusion protein.

In some embodiments, the composition includes a polypeptide molecule wherein the Par-4 polypeptide portion is a polypeptide comprising the sequence of SEQ ID NO: 1 or SEQ ID NO: 2. In some embodiments, the composition includes a polypeptide molecule wherein the Par-4 polypeptide portion is a polypeptide comprising a modification relative to the sequence of SEQ ID NO: 1 or SEQ ID NO: 2. In some embodiments, the composition includes a polypeptide molecule wherein the Par-4 polypeptide portion is a polypeptide comprising a deletion relative to the sequence of SEQ ID NO: 1 or SEQ ID NO: 2. In some embodiments, the composition includes a polypeptide molecule wherein the Par-4 polypeptide portion is a polypeptide comprising an insertion relative to the sequence of SEQ ID NO: 1 or SEQ ID NO: 2. In some embodiments, the composition includes a polypeptide molecule wherein the Par-4 polypeptide portion is a polypeptide comprising a substitution relative to the sequence of SEQ ID NO: 1 or SEQ ID NO: 2.

In some embodiments, the composition includes a polypeptide molecule wherein the Par-4 polypeptide component is a polypeptide comprising a fragment of the sequence of SEQ ID NO: 1 or SEQ ID NO: 2. In some embodiments, the composition includes a polypeptide molecule wherein the Par-4 polypeptide component is a polypeptide comprising a variant of the sequence of SEQ ID NO: 1 or SEQ ID NO: 2.

In some embodiments, the composition includes a polypeptide molecule wherein the Par-4 polypeptide portion includes a sequence having at least 85%, 86%, 87%, 88%, 89%, 90%, 91%, 92%, 93%, 94%, 95%, 96%, 97%, 98%, or 99% homology to the sequence of SEQ ID NO: 1 or SEQ ID NO: 2. In some embodiments, the composition includes a polypeptide molecule wherein the polypeptide molecule includes a Par-4 polypeptide portion including a fragment of the sequence of SEQ ID NO: 1 or SEQ ID NO: 2 having 100% homology to the sequence of SEQ ID NO: 1 or SEQ ID NO: 2 in a region including a selective for apoptosis in cancer cells (SAC) core domain. In some embodiments, the composition includes a polypeptide molecule wherein the polypeptide molecule includes a Par-4 polypeptide portion including a fragment of the sequence of SEQ ID NO: 1 or SEQ ID NO: 2 wherein 1, 2, 3, 4, 5, 6, 7, 8, 9, or 10 residues have been deleted relative to the sequence of SEQ ID NO: 1 or SEQ ID NO: 2.

As noted herein above, in addition to the Par-4 polypeptide portion, the polypeptide molecule of the compositions as disclosed herein includes a second polypeptide molecule, provided with the Par-4 polypeptide portion a as a fusion protein.

In some embodiments, the composition includes a polypeptide molecule wherein the second polypeptide molecule comprises a fragment crystallizable of immunoglobulin G1(Fc) polypeptide comprising the sequence of SEQ ID NO: 3 or SEQ ID NO: 4. In some embodiments, the composition includes a polypeptide molecule wherein the second polypeptide molecule is a polypeptide comprising a modification relative to the sequence of SEQ ID NO: 3 or SEQ ID NO: 4. In some embodiments, the composition includes a polypeptide molecule wherein the second polypeptide molecule is a polypeptide comprising a deletion relative to the sequence of SEQ ID NO: 3 or SEQ ID NO: 4. In some embodiments, the composition includes a polypeptide molecule wherein the second polypeptide molecule is a polypeptide comprising a insertion relative to the sequence of SEQ ID NO: 3 or SEQ ID NO: 4. In some embodiments, the composition includes a polypeptide molecule wherein the second polypeptide molecule is a polypeptide comprising a substitution relative to the sequence of SEQ ID NO: 3 or SEQ ID NO: 4.

In some embodiments, the composition includes a polypeptide molecule wherein the second polypeptide molecule is a fragment the sequence of SEQ ID NO: 3 or SEQ ID NO: 4. In some embodiments, the composition includes a polypeptide molecule wherein the second polypeptide molecule is a variant the sequence of SEQ ID NO: 3 or SEQ ID NO: 4.

In some embodiments, the composition includes a polypeptide molecule wherein the second polypeptide molecule including a sequence having at least 85%, 86%, 87%, 88%, 89%, 90%, 91%, 92%, 93%, 94%, 95%, 96%, 97%, 98%, or 99% homology to the sequence of SEQ ID NO: 3 or SEQ ID NO: 4. In some embodiments, the composition includes a polypeptide molecule wherein the second polypeptide molecule includes a fragment of the sequence of SEQ ID NO: 3 or SEQ ID NO: 4, wherein 1, 2, 3, 4, 5, 6, 7, 8, 9, or 10 residues have been deleted relative to the sequence of SEQ ID NO: 3 or SEQ ID NO: 4. In some embodiments, the composition includes a polypeptide molecule wherein the second polypeptide molecule includes a variant of SEQ ID NO: 3 or SEQ ID NO: 4, wherein cysteine residues of the polypeptide have been mutated relative to the sequence of SEQ ID NO: 3 or SEQ ID NO: 4. In some embodiments, the composition includes a polypeptide molecule wherein the second polypeptide molecule includes a variant of a fragment of SEQ ID NO: 3 or SEQ ID NO: 4, wherein cysteine residues of the polypeptide have been mutated relative to the sequence of SEQ ID NO: 3 or SEQ ID NO: 4, and wherein 1, 2, 3, 4, 5, 6, 7, 8, 9, or 10 residues have been deleted relative to the sequence of SEQ ID NO: 3 or SEQ ID NO: 4. In some embodiments, the composition includes a polypeptide molecule wherein the second polypeptide molecule includes a variant of SEQ ID NO: 3 or SEQ ID NO: 4 comprising the substitutions C6S, C12S, and C15S. In some embodiments, the composition includes a polypeptide molecule wherein the second polypeptide molecule includes a variant of a fragment SEQ ID NO: 3 or SEQ ID NO: 4 comprising the substitutions C6S, C12S, and C15S, and wherein 1, 2, 3, 4, 5, 6, 7, 8, 9, or 10 residues have been deleted relative to the sequence of SEQ ID NO: 3 or SEQ ID NO: 4.

In some embodiments, the composition includes a polypeptide molecule as disclosed herein, wherein the second polypeptide molecule as disclosed herein is positioned proximal to the N-terminus of an embodiment of the Par-4 polypeptide portion as disclosed herein.

In some embodiments, the composition includes a polypeptide molecule wherein the polypeptide molecule as disclosed herein comprises the sequence of SEQ ID NO: 6. In some embodiments, the polypeptide molecule as disclosed herein comprises the sequence of SEQ ID NO: 8.

The presently-disclosed subject matter also includes a method of inducing apoptosis in a cancer cell, which involves contacting the cancer cell with a composition as disclosed herein. In some embodiments, the cancer cell is metastatic.

The presently-disclosed subject matter also includes a method of treating cancer in a subject, which involves administering a composition as disclosed herein to a subject in need thereof.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this application.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

Par-4Ex protein design. Design of a desirable Par-4Ex as a therapeutic candidate must account for few issues. For example, the molecular weight of a desirable Par-4Ex must be significantly larger than that of Par-4 (˜40 kDa). For another example, the extra amino-acid residues of the extended protein could impact binding with GRP78 and, hence, make the extended protein (Par-4Ex) inactive against cancer cells. With such issue in mind and considering that the SAC domain is closer to the C-terminus, the present inventors added the extra amino-acid residues to the N-terminus of Par-4 for studies as described in these examples.

Further, with a view toward prolonging the biological half-life of Par-4, it is desirable to avoid the possible immunogenicity of the extended protein (Par-4Ex) for human. For this reason, the present inventors selected the extra amino-acid residues from a human protein fragment, but without the unnecessary biological function of the human protein. The first human protein fragment candidate in was the first 233 amino-acid residues, known as the fragment crystallizable (Fc), of human immunoglobulin G1 (IgG1). In fact, protein fusion with the Fc region of human IgG1 (IgG1 Fc) is one of the most popularly used strategies to prolong the biological half-lives of protein therapeutics, (25,26) although recent studies did reveal that the Fc fusion did not improve the biological half-life of a protein drug candidate expressed in E. coli. (27)

Wild-type IgG1 Fc will form a dimer through intermolecular disulfide bonds. Thus, the dimerization of Par-4Ex could block the interface of the intermolecular binding between Par-4 and GRP78 and, therefore, risk losing the binding affinity of Par-4 with GRP78. With this concern in mind, three cystine residues (#6, #12, and #15) of the Fc region are all changed to serine residues to avoid the possible dimerization of Par-4Ex. For convenience, the mutant Fc is denoted as Fc(M1) which refers to the first mutant (mutant 1 or M1) of Fc tested in this study. Accordingly, the first Par-4Ex protein tested in this study may also be denoted as Fc(M1)-Par-4.

Par-4 Peptide Sequences used in the Par-4Ex fusion protein and the fragment crystallizable of immunoglobulin G1 (Fc) polypeptide sequences used in the Par-4Ex fusion protein are set forth in the sequence listing. Exemplary sequences, both nucleotide and protein, which retain beneficial function of binding to GRP78 are set forth in SEQ ID. NO 5-8.

Cloning, expression and purification of 6×His-Par-4 and Par-4Ex. Bacterial expression constructs for TRX-Par-4, 6×His-Par-4, and Par-4Ex were produced by subcloning each gene into pET-22b(+) vector. The construct TRX-Par-4 was prepared by subcloning rat Par-4 sequence in frame with thioredoxin cDNA (TRX) in vector pThio-His (Invitrogen Corporation, Carlsbad, Calif.) as described in a previous report by Burikhanov et al. (2) E. coli BL21 (DE3) Star™ cells (Thermo Fisher Scientific, Waltham, Mass.) were transformed with each construct and induced with 0.5 mM IPTG (Sigma-Aldrich, St. Louis, Mo.). The cells were harvested 10 h after IPTG induction. The cells were then washed with Tris-buffered saline (25 mM Tris base, pH 7.4, 138 mM NaCl, and 2.7 mM KCl), followed by a centrifugation at 2,000×g for 5 min at 4° C. The cell homogenates were prepared through resuspending the cell pellets in 25 mM Tris-Cl, pH 7.4 and were subjected to sonication. In order to remove cell debris or unbroken cells, the total cell homogenates were exposed to centrifugation at 10,000×g for 20 min.

For purification of TRX-Par-4 or 6×His-Par-4 protein, the resultant supernatant was loaded onto a HisPur™ Cobalt Resin (Thermo Fisher Scientific) which had been pre-equilibrated with a washing buffer (25 mM Tris, pH 7.4, 500 mM NaCl, 0.05% Triton X-100, and 50 mM imidazole). After extensive washing with the washing buffer, bound His-tagged proteins were eluted by stepwise gradient elution with imidazole in the presence of 150 mM NaCl.

For purification of Par-4Ex protein, the resultant supernatant was loaded onto a rmp Protein A Sepharose Fast Flow (GE Healthcare Life Sciences) pre-equilibrated with 20 mM Tris.HCl (pH 7.4). Then, the mixture was packed in a column and washed with 5 column volume (CV) of 20 mM Tris.HCl (pH 7.4) containing 200 mM NaCl until an OD₂₈₀<0.02 was achieved. Then, the protein was eluted by 50 mM sodium acetate, pH 4.0, containing 200 mM NaCl. For buffer exchange, the eluate was dialyzed in 20 mM Tris.HCl (pH 7.4) by Millipore Centrifugal Filter Units. The protein solution was then loaded onto a Q-Sepharose Fast Flow (GE Healthcare Life Sciences, Pittsburgh, Pa.) pre-equilibrated with 20 mM Tris.HCl, pH 7.4, for the second-round chromatographic separation. Par-4Ex protein was eluted from the Q-Sepharose column with a stepwise gradient of NaCl (100-800 mM). For buffer exchange, the eluate was dialyzed in storage buffer (50 mM Hepes, 20% sorbitol, 1 M glycine, pH 7.4) by Millipore Centrifugal Filter Units. The entire purification process was performed in a cold room at 8° C. and the purified proteins were stored at −80° C. until the activity tests. Their purity was analyzed by SDS-PAGE on a 4-12% NuPAGE Novex Bis-Tris gel (Life Technologies).

Western blotting. Recombinant proteins in bacterial extract transformed with pET-22b(+)/6×his-Par-4 or pET-22b(+)/Par-4Ex were analyzed by western blot using goat anti-rat Par-4 IgG obtained from Santa Cruz Biotechnology (Dallas, Tex.) (1:3000 dilution as described in the manufacturer's instructions). Pre-adsorbed, HRP-conjugated anti-goat IgG (Santa Cruz Biotechnology) was used at 1:4000 as a secondary antibody and Par-4 protein was finally detected by chemiluminescence using the SuperSignal West Dura Extended Duration Substrate from Pierce Biotechnology (Waltham, Mass.).

Immunocytochemistry and apoptosis analysis. Cells in chamber slides were exposed to 100 nM purified Par-4Ex or 6×His-Par-4. 24 h after treatment, the cells were subjected to immunocytochemistry (ICC) using the indicated anti-caspase 3 IgG and then stained with the appropriate secondary antibody conjugated to Alexa Fluor-488 (green fluorescence) or Alexa Fluor-594 (red fluorescence) (Molecular Probes). Apoptotic nuclei were identified by TUNEL assay, caspase-3 immunostaining, or 4,6-diamidino-2-phenylindole (DAPI) staining. A total of three independent experiments were performed, and approximately 300 cells were scored in each experiment for apoptosis under a fluorescent microscope, as described previously. (28)

Pharmacokinetic studies in mice. Mice were injected with each recombinant protein or saline through the tail vein at a dose of 5 mg/kg of body weight. Blood samples (15-30 μL, each) were collected from saphenous veins into heparin-treated capillary tubes at various time points after intravenous (i.v.) administration of the protein. The plasma was separated from the collected blood by centrifugation (15 min, at 5,000×g). 200 ng of plasma protein in 100 μL 0.05 M PBS, pH 7.4, was immobilized in a 96 well flat-bottomed EIA plate (Corning) at 4° C. overnight (or 37° C. for 2 h). The liquid was dumped from the plates and the rest was drained on paper towel. Coated wells were blocked with blocking buffer (0.05 M PBS, pH 7.4, containing 1 mg/ml casein) (250 μL/well) at RT for 30 min. After washing twice with washing buffer (0.05 M PBS, pH 7.4) (250 μL/well), 100 μL of goat anti-rat Par-4 IgG (Santa Cruz Biotechnology) in blocking buffer was added to each well at a range of concentrations. The plate was then covered with an adhesive plastic and incubated, with continual shaking, at RT for 1 h. After washing three times with washing buffer, pre-adsorbed, HRP-conjugated secondary antibody (anti-goat IgG-HRP) (70 μL/well), diluted with blocking buffer at a ratio of 1:30,000, was added into each well and incubated at RT for 1 h on a shaker. The wells were then washed for three times with washing buffer (250 μL/well) before 250 μL TMB substrate was added to the wells. The ELISA plate was kept in the dark until the desired color developed. The reaction was stopped with 100 μL, of 0.5 M HCl. The absorbance (=the developed blue color) was measured at 450 nm using a microplate reader. All measurements were performed in triplicate or quadruplicate. The obtained PK data (time dependent enzyme concentrations) ([E]t) were fitted to a double-exponential equation (29) by GraphPad Prism 5.01 software: [E]ₜ=Ae^{-k}^{₁}^{t}+Be^{-k}^{₂}^{t}, which accounts for both the distribution process (the fast phase, associated with k₁) and the elimination process (the slow phase, associated with k₂) of the protein in animals. The t_{1/2} associated with the elimination rate constant k₂ of the fusion protein is known as the biological tin or elimination tin.

Lung metastasis of breast cancer. The E0771 cells (1.5×10⁵ cells) were administered into tail veins in immunodeficient B6C3H mice (n=5). 5 h after administration, 6×His-Par-4 or Par-4Ex was injected (i.v.) through tail vein every alternate day in the dose of 250 or 150 or 75 μg/injection for each mouse within 12 days (for a total of 1500 or 900 or 450 μg of protein/mouse during the 12 days). Four weeks later, the mice were euthanized, and the lungs were photographed. The number of the lung nodules were then counted.

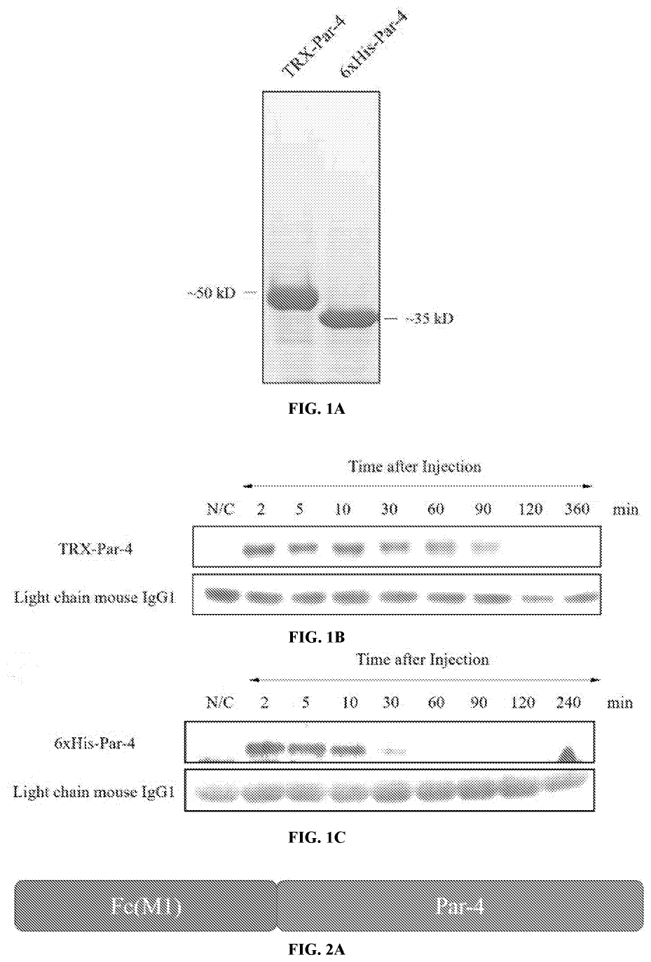

Results. Pharmacokinetics of recombinant Par-4. In a previous report by Zhao et al., (15) it was demonstrated that the i.v. administration of recombinant TRX-Par-4 or TRX-SAC protein (prepared in E. coli) in immunocompetent C57/BL6 mice significantly suppressed lung metastasis of LLC1 (Lewis lung carcinoma line 1) cells in mice, (15) which implies that both extracellular Par-4 and SAC proteins are capable of inhibiting metastatic tumor growth in vivo. Thioredoxin (TRX) fusion protein is a frequently used tool to increase the solubility and expression of mammalian proteins when they are expressed heterologously in E. coli. (30) However, the pharmacokinetic profiles of TRX-Par-4 and TRX-SAC proteins have not yet been determined despite of considerable in vitro and in vivo anti-tumor activity assays against various cancer cells. (2,15,17) Therefore, it was determined how long TRX-Par-4 or unfused Par-4 protein can stay in the circulatory system of mice. To address this question, Par-4 protein fused to the C-terminus of TRX or 6×His-tag was prepared using the bacterial expression system (TRX-Par-4 and 6×His-Par-4, respectively; see FIG. 1A), and each purified protein was then injected to mice at a dose of 5 mg/kg i.v. through tail vein. Blood samples were collected at varying time points after the protein injection and analyzed by Western blotting using anti-Par-4 antibody. The results revealed that both TRX-Par-4 (˜50 kDa) and 6×His-Par-4 (˜38 kDa) proteins are quickly removed from the circulatory system (FIGS. 1B & C). TRX-Par-4 concentration decreased in vivo at a relatively slower than that of 6×His-Par-4, with the former able to be observed up to 90 min after i.v. injection, while 6×His-Par-4 detected at very low signal up to 30 min. These findings suggest the possibility that the in vivo anti-tumor activity of Par-4 protein may be limited due to its short circulating half-life.

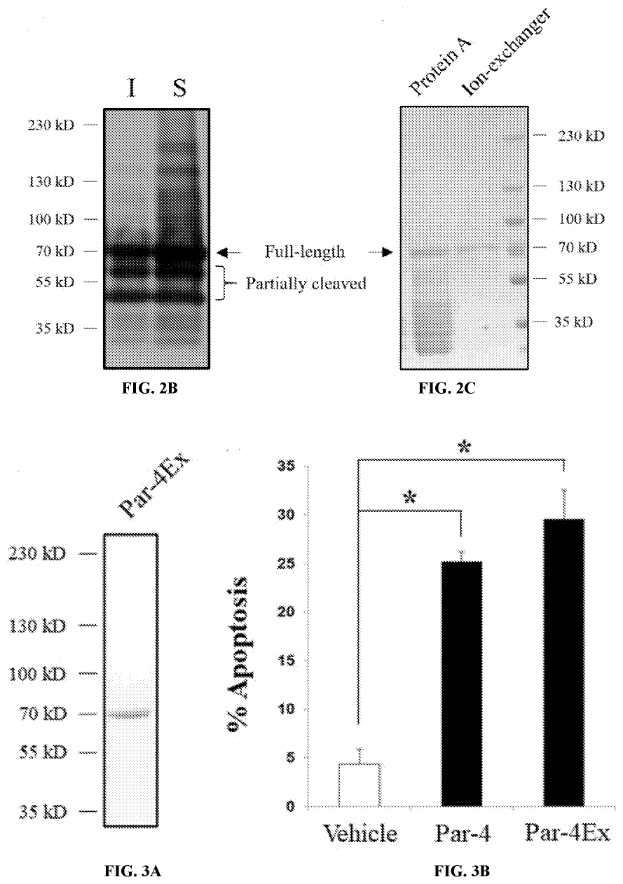

Par-4 and Par-4Ex proteins expressed in E. coli similarly induced apoptosis in cancer cells. Along with the preparation of the TRX-Par-4 and unfused Par-4 proteins, fusion protein Par-4Ex, i.e. Fc(M1)-Par-4 (˜70 kDa), was also prepared using the E. coli expressing system to obtain the amount of protein sufficient for the in vivo characterization study of the protein. Purification of the E. coli-derived soluble Par-4Ex protein was performed using protein A affinity chromatography, followed by an additional ion-exchange chromatographic step to obtain the purity required for a following in vivo characterization study (FIGS. 2B & C).

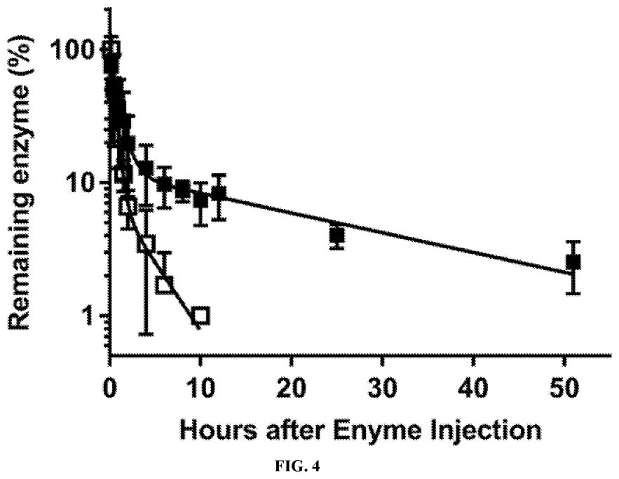

Before in vivo testing of the purified recombinant Par-4 and Par-4Ex proteins, a determination of whether Par-4Ex retains the unique proapoptotic activity of Par-4 was desired because the Fc(M1) fusion to the N-terminus of Par-4 could block or interfere the protein-protein interactions between and Par-4 and GRP78. To address this crucial question, E0771 (murine breast cancer cell line) cells were treated with either 100 nM 6×His-Par-4 or Par-4Ex protein, followed by incubation for 24 h. Storage buffer (50 mM Hepes, 20% sorbitol, 1 M glycine, pH 7.4) was used as a vehicle control. It was observed that Par-4Ex and 6×His-Par-4 proteins induced a similar level of apoptosis in E0771 cells in a given treatment condition (FIG. 3), demonstrating that the anti-tumor activity of Par-4 protein is not altered by the Fc(M1) fusion to its N-terminus. This observation of the anti-tumor activity of Par-4 protein itself is consistent with the findings of Zhao et al. (15) using TRX-Par-4. The study further demonstrated that Par-4Ex was as active as Par-4 itself in the cell-based anti-tumor activity assays.

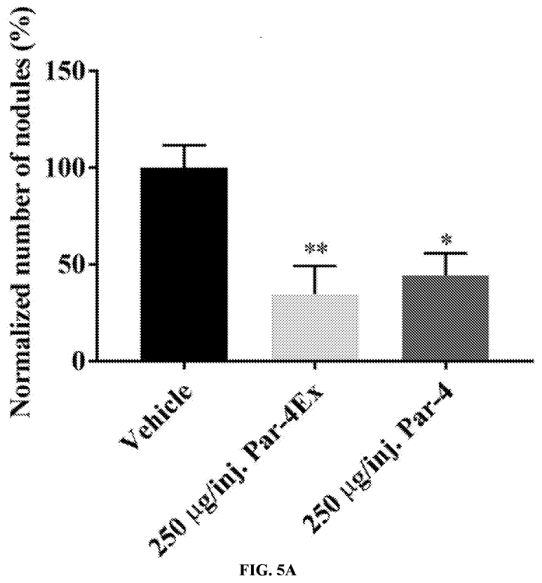

Biological half-lives of recombinant proteins. To examine whether Par-4Ex really has a prolonged biological half-life compared to Par-4, a pharmacokinetic (PK) study was carried out in mice. The in vivo data were based on intravenous (i. v.) injection of each protein in the tested mice. The generated PK data are depicted in FIG. 4. The results revealed that the Par-4Ex has a much longer biological half-life (˜20.3 h) compared to that (˜3 h) of the 6×His-Par-4 protein.

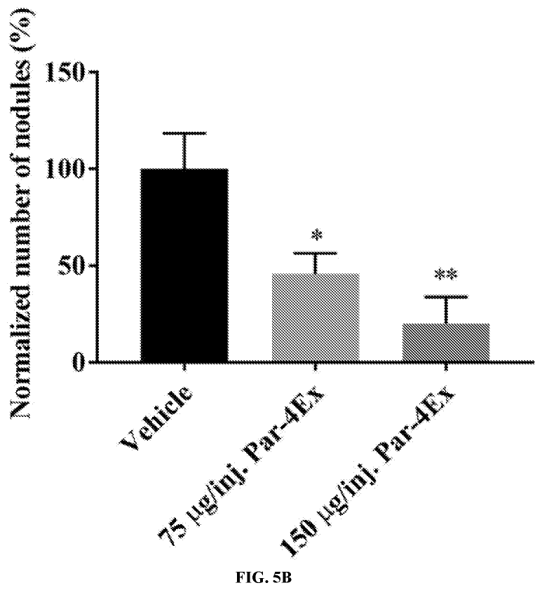

In vivo potency in inhibiting metastatic tumor growth. To examine whether the longer exposure due to the biological half-life extension improves the in vivo anti-tumor activity of Par-4, it was evaluated how efficiently Par-4Ex and 6×His-Par-4 proteins inhibit lung metastasis of E0771 breast cancer cells in immunocompetent C57/BL6 mice. The cells (1.5×10⁵ cells) were injected through tail vein, and then E. coli-derived Par-4Ex (250, 150 or 75 μg) or 6×His-Par-4 (250 μg) was administered intravenously every other day for 12 days (for a total of 1500 or 900 or 450 μg per mouse within 12 days). It was observed that both Par-4Ex and 6×His-Par-4 proteins significantly suppressed metastatic tumor growth in vivo compared to vehicle-treated control, but with no statistic difference between the two protein-treated groups in a given treatment condition (FIG. 5A). However, considering that the molecular weight of Par-4Ex protein (˜70 kDa) is approximately 1.8-fold higher than that of 6×His-Par-4 protein (˜38 kDa), the mice have not been treated with equivalent molar concentrations of recombinant Par-4 proteins. This may suggest that Par-4Ex was indeed more potent than 6×His-Par-4 in inhibiting metastatic tumor growth in vivo, but the difference in their potency might become less evident at the high dose. To more appropriately address this question, it was determined whether the in vivo anti-tumor activity of Par-4Ex is diminished with decreasing the dose. According to the data obtained (FIG. 5B), a 70%-reduced dose of Par-4Ex protein (75 μg/injection) also induced substantial inhibition of lung metastasis in mice induced by E0771 breast cancer cells. In comparison, previous studies (15) examined various doses of the Par-4 (TRX-Par-4) for its in vivo activity using the same animal model and found that 250 μg/injection was the minimum effective dose; lung metastasis by E0771 breast cancer cells was not significantly reduced by the Par-4 injection at any of the tested doses lower than 250 μg/injection. Taking all of these together, Par-4Ex indeed has an improved in vivo potency than Par-4 in inhibiting metastatic tumor growth.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Sells S F, Wood D P, Jr., Joshi-Barve S S, Muthukumar S, Jacob R J, Crist S A, et al. Commonality of the gene programs induced by effectors of apoptosis in androgen-dependent and -independent prostate cells. Cell Growth Differ 1994; 5(4):457-66.
2. Burikhanov R, Zhao Y, Goswami A, Qiu S, Schwarze S R, Rangnekar V M. The tumor suppressor Par-4 activates an extrinsic pathway for apoptosis. Cell 2009; 138(2):377-88 doi 10.1016/j.cell.2009.05.022.
3. Gurumurthy S, Rangnekar V M. Par-4 inducible apoptosis in prostate cancer cells. J Cell Biochem 2004; 91(3):504-12 doi 10.1002/jcb.20000.
4. Moreno-Bueno G, Fernandez-Marcos P J, Collado M, Tendero M J, Rodriguez-Pinilla SM, Garcia-Cao I, et al. Inactivation of the candidate tumor suppressor par-4 in endometrial cancer. Cancer Res 2007; 67:1927-34.
5. Cook J, Krishnan S, Ananth S, Sells S F, Shi Y, Walther M M, et al. Decreased expression of the pro-apoptotic protein Par-4 in renal cell carcinoma. Oncogene 1999; 18:1205-8.
6. Alvarez J V, Pan T C, Ruth J, Feng Y, Zhou A, Pant D, et al. Par-4 downregulation promotes breast cancer recurrence by preventing multinucleation following targeted therapy. Camcer Cell 2013; 24:30-44.
7. Mabe N W, Fox D B, Lupo R, Decker A E, Phelps S N, Thompson J W, et al. Epigenetic silencing of tumor suppressor Par-4 promotes chemoresistance in recurrent breast cancer. J Clin Invest 2018; 128:4413-28.
8. Kogel D, Reimertz C, Mech P, Poppe M, Fruhwald M C, Engemann H, et al. Dlk/ZIP kinase-induced apoptosis in human medulloblastoma cells: requirement of the mitochondrial apoptosis pathway. Br J Cancer 2001; 85(11):1801-8 doi 10.1054/bjoc.2001.2158.
9. Liu Y, Gilbert M R, Kyprianou N, Rangnekar V M, Horbinski C. The tumor suppressor prostate apoptosis response-4 (Par-4) is regulated by mutant IDH1 and kills glioma stem cells. Acta Neuropathol 2014; 128:723-32.
10. Oda S, Kato Y, Hatakeyama M, Iwamura A, Fukami T, Kume T, et al. Evaluation of expression and glycosylation status of UGT1A10 in Supersomes and intestinal epithelial cells with a novel specific UGT1A10 monoclonal antibody. Drug Metab Dispos 2017; 45(9):1027-34 doi 10.1124/dmd.117.075291.
11. El-Guendy N, Rangnekar V M. Apoptosis by Par-4 in cancer and neurodegenerative diseases. Exp Cell Res 2003; 283(1):51-66.
12. Hebbar N, Wang C, Rangnekar V M. Mechanisms of apoptosis by the tumor suppressor Par-4. J Cell Physiol 2012; 227(12):3715-21 doi 10.1002/jcp.24098.
13. Chakraborty M, Qiu S G, Vasudevan K M, Rangnekar V M. Par-4 drives trafficking and activation of Fas and Fasl to induce prostate cancer cell apoptosis and tumor regression. Cancer Res 2001; 61(19):7255-63.
14. El-Guendy N, Zhao Y, Gurumurthy S, Burikhanov R, Rangnekar V M. Identification of a unique core domain of par-4 sufficient for selective apoptosis induction in cancer cells. Mol Cell Biol 2003; 23(16):5516-25.
15. Zhao Y, Burikhanov R, Brandon J, Qiu S, Shelton B J, Spear B, et al. Systemic Par-4 inhibits non-autochthonous tumor growth. Cancer Biol Ther 2011; 12(2):152-7.
16. Burikhanov R, Sviripa V M, Hebbar N, Zhang W, Layton W J, Hamza A, et al. Arylquins target vimentin to trigger Par-4 secretion for tumor cell apoptosis. Nat Chem Biol 2014; 10(11):924-6 doi 10.1038/nchembio.1631.
17. Burikhanov R, Hebbar N, Noothi S K, Shukla N, Sledziona J, Araujo N, et al. Chloroquine-Inducible Par-4 Secretion Is Essential for Tumor Cell Apoptosis and Inhibition of Metastasis. Cell Rep 2017; 18(2):508-19 doi 10.1016/j.celrep.2016.12.051.
18. Putney S D, Burke P A. Improving protein therapeutics with sustained-release formulations. Nat Biotechnol 1998; 16(2):153-7 doi 10.1038/nbt0298-153.
19. Bandaranayake A D, Almo S C. Recent advances in mammalian protein production. FEBS letters 2014; 588(2):253-60 doi 10.1016/j.febslet.2013.11.035.
20. Butler M, Meneses-Acosta A. Recent advances in technology supporting biopharmaceutical production from mammalian cells. Appl Microbiol Biotechnol 2012; 96(4):885-94 doi 10.1007/s00253-012-4451-z.
21. Zhu J. Mammalian cell protein expression for biopharmaceutical production. Biotechnol Adv 2012; 30(5):1158-70 doi 10.1016/j.biotechadv.2011.08.022.
22. Tobin P H, Richards D H, Callender R A, Wilson C J. Protein engineering: a new frontier for biological therapeutics. Curr Drug Metab 2014; 15(7):743-56.
23. Wurm F M. Production of recombinant protein therapeutics in cultivated mammalian cells. Nat Biotechnol 2004; 22(11):1393-8 doi 10.1038/nbt1026.
24. Lutz S. Beyond directed evolution—semi-rational protein engineering and design. Curr Opin Biotechnol 2010; 21(6):734-43 doi 10.1016/j.copbio.2010.08.011.
25. Levin D, Golding B, Strome S E, Sauna Z E. Fc fusion as a platform technology: potential for modulating immunogenicity. Trends Biotechnol 2015; 33(1):27-34 doi 10.1016/j.tibtech.2014.11.001.
26. Rath T, Baker K, Dumont J A, Peters R T, Jiang H, Qiao S W, et al. Fc-fusion proteins and FcRn: structural insights for longer-lasting and more effective therapeutics. Crit Rev Biotechnol 2015; 35(2):235-54 doi 10.3109/07388551.2013.834293.
27. Chen X, Xue L, Hou S, Jin Z, Zhang T, Zheng F, et al. Long-acting cocaine hydrolase for addiction therapy. Proc Natl Acad Sci USA 2016; 113:422-7.
28. Goswami A, Burikhanov R, de Thonel A, FujitaN, Goswami M, Zhao Y, et al. Binding and phosphorylation of par-4 by akt is essential for cancer cell survival. Mol Cell 2005; 20(1):33-44 doi 10.1016/j.molcel.2005.08.016.
29. Lockridge O, Schopfer L M, Winger G, Woods J H. Large Scale Purification of Butyrylcholinesterase from Human Plasma Suitable for Injection into Monkeys; a Potential New Therapeutic for Protection against Cocaine and Nerve Agent Toxicity. J Med Chem Biol Radiol Def 2005; 3:nihms5095 doi 10.1901/j aba.2005.3-nihms5095.
30. Berndt C, Lillig C H, Holmgren A. Thioredoxins and glutaredoxins as facilitators of protein folding. Biochim Biophys Acta 2008; 1783(4):641-50 doi 10.1016/j.bbamcr.2008.02.003.
31. Knop K, Hoogenboom R, Fischer D, Schubert U S. Poly(ethylene glycol) in drug delivery: pros and cons as well as potential alternatives. Angew Chem Int Ed Engl 2010; 49(36):6288-308 doi 10.1002/anie.200902672.
32. Miner J H. The glomerular basement membrane. Exp Cell Res 2012; 318(9):973-8 doi 10.1016/j.yexcr.2012.02.031.
33. Sztal T, Berger S, Currie P D, Hall T E. Characterization of the laminin gene family and evolution in zebrafish. Dev Dyn 2011; 240(2):422-31 doi 10.1002/dvdy.22537.
34. Suh J H, Jarad G, VanDeVoorde R G, Miner J H. Forced expression of laminin beta1 in podocytes prevents nephrotic syndrome in mice lacking laminin beta2, a model for Pierson syndrome. Proc Natl Acad Sci USA 2011; 108(37):15348-53 doi 10.1073/pnas.1108269108.
35. Hausmann R, Kuppe C, Egger H, Schweda F, Knecht V, Eiger M, et al. Electrical forces determine glomerular permeability. J Am Soc Nephroi 2010; 21(12):2053-8 doi 10.1681/ASN.2010030303.
36. Czajkowsky D M, Hu J, Shao Z, Pleass R J. Fc-fusion proteins: new developments and future perspectives. EMBO Mol Med 2012; 4(10):1015-28 doi 10.1002/emmm.201201379.
37. Kenanova V E, Olafsen T, Salazar F B, Williams L E, Knowles S, Wu A M. Tuning the serum persistence of human serum albumin domain III: diabody fusion proteins. Protein Eng Des Sel 2010; 23(10):789-98 doi 10.1093/protein/gzq054.
38. Carter J, Zhang J, Dang T L, Hasegawa H, Cheng J D, Gianan I, et al. Fusion partners can increase the expression of recombinant interleukins via transient transfection in 2936E cells. Protein Sci 2010; 19(2):357-62 doi 10.1002/pro.307.
39. Sockolosky J T, Szoka F C. The neonatal Fc receptor, FcRn, as a target for drug delivery and therapy. Adv Drug Deliv Rev 2015; 91:109-24 doi 10.1016/j.addr.2015.02.005.
40. Kuo T T, Aveson V G. Neonatal Fc receptor and IgG-based therapeutics. MAbs 2011; 3(5):422-30 doi 10.4161/mabs.3.5.16983.
41. Mankarious S, Lee M, Fischer S, Pyun K H, Ochs H D, Oxelius V A, et al. The half-lives of IgG subclasses and specific antibodies in patients with primary immunodeficiency who are receiving intravenously administered immunoglobulin. J Lab Clin Med 1988; 112(5):634-40.
42. Correia I R. Stability of IgG isotypes in serum. MAbs 2010; 2(3):221-32.
43. Oganesyan V, Damschroder M M, Cook K E, Li Q, Gao C, Wu H, et al. Structural insights into neonatal Fc receptor-based recycling mechanisms. J Biol Chem 2014; 289(11):7812-24 doi 10.1074/jbc.M113.537563.
44. Schmidt M M, Townson S A, Andreucci A J, King B M, Schirmer E B, Murillo A J, et al. Crystal structure of an HSA/FcRn complex reveals recycling by competitive mimicry of HSA ligands at a pH-dependent hydrophobic interface. Structure 2013; 21(11):1966-78 doi 10.1016/j.str.2013.08.022.
45. Christianson G J, Sun V Z, Akilesh S, Pesavento E, Proetzel G, Roopenian D C. Monoclonal antibodies directed against human FcRn and their applications. MAbs 2012; 4(2):208-16 doi 10.4161/mabs.4.2.19397.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A composition, comprising:
a prostate apoptosis response-4 (Par-4) polypeptide comprising
a sequence having at least 90% homology to the sequence of SEQ ID NO: 1 or SEQ ID NO: 2, or
a fragment thereof having 100% homology to the sequence of SEQ ID NO: 1 or SEQ ID NO: 2 in a region including a selective for apoptosis in cancer cells (SAC) core domain; and
a second polypeptide molecule,
wherein the Par-4 polypeptide and the second polypeptide molecule are provided as a fusion protein.

2. The composition of claim 1, wherein 1, 2, 3, 4, 5, 6, 7, 8, 9, or 10 residues have been removed relative to the sequence of SEQ ID NO: 1 or SEQ ID NO: 2

3. The composition of claim 1, comprising a sequence selected from the sequences consisting of SEQ ID NO: 6 and 8.

4. The composition of claim 1, wherein the fusion protein comprises a fragment crystallizable of immunoglobulin G1(Fc) polypeptide comprising the sequence of SEQ ID NO: 3 or SEQ ID NO: 4, or a fragment thereof wherein 1, 2, 3, 4, 5, 6, 7, 8, 9, or 10 residues have been removed relative to the sequence of SEQ ID NO: 3 or SEQ ID NO: 4, or a variant thereof wherein cysteine residues of the polypeptide have been mutated relative to the sequence of SEQ ID NO: 3 or SEQ ID NO: 4.

5. The composition of claim 4, wherein the Fc polypeptide is positioned proximal to the N-terminus of the Par-4 polypeptide.

6. The composition of claim 5, wherein the Fc polypeptide comprises the mutations: C6S, C12S, and C15S.

7. A method of inducing apoptosis in a cancer cell, comprising: contacting the cancer cell with the composition of claim 1.

8. The method of claim 7, wherein the cancer cell is metastatic.

9. A polypeptide molecule, comprising:
a prostate apoptosis response-4 (Par-4) polypeptide comprising
a sequence having at least 90% homology to the sequence of SEQ ID NO: 1 or SEQ ID NO: 2, or
a fragment thereof having 100% homology to the sequence of SEQ ID NO: 1 or SEQ ID NO: 2 in a region including a selective for apoptosis in cancer cells (SAC) core domain; and
a second polypeptide molecule,
wherein the Par-4 polypeptide and the second polypeptide molecule are provided as a fusion protein.

10. The polypeptide of claim 9, wherein 1, 2, 3, 4, 5, 6, 7, 8, 9, or 10 residues have been deleted relative to the sequence of SEQ ID NO: 1 or SEQ ID NO: 2.

11. The polypeptide molecule of claim 10, comprising a sequence selected from the sequences consisting of SEQ ID NO: 6 and 8.

12. The polypeptide molecule of claim 10, wherein the fusion protein comprises a fragment crystallizable of immunoglobulin G1(Fc) polypeptide comprising the sequence of SEQ ID NO: 3 or SEQ ID NO: 4, or a fragment thereof wherein 1, 2, 3, 4, 5, 6, 7, 8, 9, or 10 residues have been deleted relative to the sequence of SEQ ID NO: 3 or SEQ ID NO: 4, or a variant thereof wherein cysteine residues of the polypeptide have been mutated relative to the sequence of SEQ ID NO: 3 or SEQ ID NO: 4.

13. The polypeptide molecule of claim 12, wherein the Fc polypeptide is positioned proximal to the N-terminus of the Par-4 polypeptide.

14. The polypeptide molecule of claim 13, wherein the Fc polypeptide comprises the substitutions consisting of: C6S, C12S, and C15S.

15. A method of inducing apoptosis in a cancer cell, comprising: contacting the cancer cell with the polypeptide molecule of claim 9.

16. The method of claim 15, wherein the cancer cell is metastatic.

17. A nucleic acid molecule, comprising: a nucleotide sequence, or complement thereof, encoding a fusion protein comprising
a prostate apoptosis response-4 (Par-4) polypeptide comprising
a sequence having at least 90% homology to the sequence of SEQ ID NO: 1 or SEQ ID NO: 2, or
a fragment thereof having 100% homology to the sequence of SEQ ID NO: 1 or SEQ ID NO: 2 in a region including a selective for apoptosis in cancer cells (SAC) core domain; and
a second polypeptide molecule.

18. The nucleic acid molecule of claim 17, comprising a sequence selected from the sequences consisting of SEQ ID NO: 5 and 7, or a complement thereof.

19. A vector, comprising the nucleic acid molecule of claim 17.

20. A cell, comprising the vector of claim 19.
